(19) United States
(12) Patent Application Publication
(10) Pub. No.: US 2024/0197650 A1
(43) Pub. Date: Jun. 20, 2024

(54) Combination of Cinnamaldheyde and Eugenol with Antimycotic Activity

(71) Applicant: TARGETING GUT DISEASE SRL, Bologna (BO) (IT)

(72) Inventor: Maria Chiara VALERII, Bologna (BO) (IT)

(21) Appl. No.: 18/288,840

(22) PCT Filed: Jun. 28, 2022
(86) PCT No.: PCT/EP2022/067681

§ 371 Date: Oct. 30, 2023

(30) Foreign Application Priority Data
Jun. 29, 2021 (IT) 102021000017054

(51) Int. Cl. A61K 31/11; A61K 31/085; A61K 36/54; A61K 36/61; A61P 31/10

(52) U.S. Cl. CPC A61K 31/11; A61K 31/085; A61K 36/54; A61K 36/61; A61P 31/10

(57) ABSTRACT

A combination of active ingredients of plant origin or synthetic analogues or extracts of plant origin containing cinnamaldheyde and eugenol or derivatives thereof, useful as antimycotic for both human and veterinary therapeutic use, mainly against fungal infections caused by Candida. Compositions containing the combination for topical or systemic administration are also described.

The present invention relates to a combination of active ingredients of plant origin, particularly a combination of active ingredients of plant origin or synthetic analogues or extracts of plant origin containing cinnamaldheyde together with eugenol or derivatives thereof, useful as antimycotic for both human and veterinary therapeutic use, mainly against fungal infections caused by Candida.

Candida spp is a yeast which may cause fungal infections in humans and animals. The most important species is Candida albicans which is present in the mucosal membranes of about 80% of the human population and is part of the normal microbial flora of skin, mouth, gastrointestinal tract and vagina.

Even if it is not normally pathogen, however, this yeast may cause infections in debilitated organisms. Such infections are typically referred to as candidosis when they affect the vaginal area or thrush when they affect the mouth.

Essential oils and their active ingredients have biological activities which, if properly used and suitably dosed, may act as germicides, fungicides, antibacterials, antiinflammatories, antiparasitics and antibiotics.

Cinnamaldheyde or cinnamic aldheyde is a substance present in the essential oil extracted from cinnamon. It showed a very good antimycotic action in several studies, in particular against several strains of Candida genus.

Eugenol is a substance present in the essential oil extracted from cloves, which showed to be effective as antimycotic, in addition to have multiple antimicrobial effects.

We have now found that the combination of cinnamaldehyde and eugenol has advantageous antimycotic effects which make it particularly useful in the treatment of fungal infections, in particular caused by Candida spp, both in human beings and in animals.

Therefore, object of the present invention is a combination of cinnamaldehyde and eugenol for use as antimycotic in humans and animals.

In the present context, the terms “cinnamaldehyde” and “eugenol” are used to refer to the single isolated and/or purified substances as well as to refer to extracts and essential oils of plant origin containing them. Similarly, the terms “cinnamaldehyde” and “eugenol”, unless otherwise stated, also include cinnamaldehyde derivatives and eugenol derivatives which maintain the same therapeutic activity.

In the literature there have been described some compositions containing mixtures of essential oils of plant origin, including essential oils containing cinnamaldehyde and eugenol.

For example, WO2012/114201 describes compositions based on carvacrol (contained in the essential oil extracted from oregano) with broad spectrum antibacterial, antiparassitic and antifungal activity. The compositions may further contain other essential oils among which essential oils containing eugenol and essential oils containing cinnamaldehyde are mentioned.

WO2004/076680 describes an antimicrobial agent of plant origin which may contain several essential oils among which also essential oils containing cinnamaldehyde and/or eugenol.

However, there is no description in the prior art of antimycotic compositions containing only the combination of cinnamaldehyde and eugenol as active ingredient.

It should also be noted that only the combination of cinnamaldehyde and eugenol shows advantages in the administration of the combination of the two active ingredients compared to the administration of the single active ingredients.

Additional essential oils known for their antimycotic activity, such as, for example, α-pinene, did not show any advantage when administered in combination with cinnamaldehyde or eugenol.

The combination of cinnamaldehyde and eugenol, object of the present invention, can be administered in the form of conventional formulations suitable to oral or topical administration. The skilled in the art is able to select the most appropriate excipients for the formulation, which selection will depend on the route of administration and the type of infection to be treated.

In fact, the combination of cinnamaldehyde and eugenol exerts its therapeutic activity topically as well as systemically.

If a systemic action is preferable or desirable, the formulation containing the combination of cinnamaldheyde and eugenol will be preferably an oral formulation, such as tablets, capsules and granulates, which can also be administered in the form of food supplements.

If a topical action is preferable or desirable, the formulation containing the combination of cinnamaldheyde and eugenol will be an oral formulation suitable for a prolonged stay within the oral cavity (e.g. candies or chewing-gums) in case of fungal infection of the oral cavity (e.g. thrush) or a formulation for topical vaginal application (e.g. vaginal suppositories or creams) in case of fungal infection at vaginal level.

The selection of the excipients/additives to be used for the formulation is within the common knowledge of the skilled in the art.

Therefore, a further object of the present invention is a composition for topical or systemic administration containing a combination of cinnamaldehyde and eugenol in admixture with a suitable carrier.

The amount of cinnamaldheyde and eugenol contained in the compositions object of the present invention varies depending on the form of administration used and preferably corresponds to a cinnamaldheyde:eugenol ratio ranging from 1:1 to 1:10 parts by weight, still more preferably cinnamaldheyde:eugenol 1:2.

The daily dose varies from a minimum of 0.03 mg/kg to a maximum of 300 mg/kg for cinnamaldheyde and from a minimum of 0.03 mg/kg to a maximum of 300 mg/kg for eugenol.

For the formulation, eugenol and cinnamaldheyde contained in essential oils such as Cinnamon Essential Oil with eugenol titer ranging from 1 to 90% or Cinnamon Essential Oil with cinnamaldheyde titer ranging from 1 to 90% or Glove Essential Oil with eugenol titer ranging from 1 to 90% or Glove Essential Oil with cinnamaldheyde titer ranging from 1 to 90% can be used. Also cinnamaldheyde and eugenol isolated and purified from essential oils or synthetic products can be used. The oral formulations can include standard or modified-release capsules, granulates o tablets, the compounds can be free, adsorbed on fibers of plant origin or synthetic, microincapsulated or nanoincapsulated.

The therapeutic advantages in administering the cinnamaldheyde+eugenol combination object of the present invention have been assessed with experimental trials described in details in the experimental part below.

The experiments confirmed in particular the antimycotic activity of cinnamaldheyde and eugenol separately and the surprisingly much more pronounced effect when administered in combination.

In fact, from the experiment on plates, the disks soaked with eugenol and cinnamaldheyde separately inhibited the growth of all tested Candida strains, causing inhibition rings notably greater than those caused by amphotericin B (10 μg). Cinnamaldehyde and eugenol showed very good inhibiting activity on fungal growth with average MIC values of 0.0048% (v/v) and 0.043% (v/v), respectively. The fungicidal activity results stronger for cinnamaldheyde (0.01% v/v) than for eugenol (0.067% v/v). At the respective fungicidal concentration, eugenol kills all the fungal cells in 1 hour, cinnamaldheyde employs more than 2 hours but less than 4 hours to completely kill the fungal cells.

Their combination has an antimycotic affect much more remarkable by lowering the eugenol MIC to ⅛ and the cinnamaldheyde MIC to ½ compared to those of the single oils.

Finally, the combination of cinnamaldheyde and eugenol, object of the present invention, showed to have no effect on Lactobacillus at the concentrations useful to inhibit Candida spp, so allowing to keep the normal bacterial flora unaltered.

### BRIEF DESCRIPTION OF THE FIGURES

FIG. 1 depicts the reading scheme of the disk diffusion method.

FIG. 2 depicts the reading scheme of the microdilution method—am=amphotericin B (UdM: μg/ml), e.o.=essential oil (UdM: % v/v), negative control: DMSO 1% v/v.

FIG. 3 depicts the incubation scheme of the time-kill curve method.

FIG. 4 shows the inhibition rings obtained with the disk diffusion method.

FIG. 5 shows the microtiter plate for C. albicans 14.

FIG. 6 shows the MFC reading (seed number 6 has no colonies, MFC=0.125% v/v).

FIG. 7 reports the time-kill curve for C. albicans strain 15. Oils used at their MFC (corresponding to 2XMIC for the C. albicans strain 15).

FIG. 8 shows the cinnamaldheyde/eugenol checkerboard (FICI 0.625).

### EXPERIMENTAL PART

### Antifungal Activity of Cinnamaldheyde and Eugenol Vs Candida Spp.

### Materials and Methods

A total of eighteen strains belonging to Candida spp. (15 C. albicans, 2 C. glabrata and 1 C. lusitaniae) derived from a collection of vaginal strains isolated in CHROMAgar Candida (BD Italia SpA, Milan, Italy) were grown on Potato Dextrose Agar (Oxoid Thermofisher SpA, Milan, Italy) at 35° C. for 24 hours.

### Antifungal Activity: Disk Diffusion Method

Colonies were re-suspended in sterile saline at a density corresponding to #0.5 McFarland (opacity standard). #0.5 McF correspond to 1.4×10\hat{ }6 colony forming units (CFU)/ml for C. albicans, 4.3×10\hat{ }6 CFU/ml for C. glabrata and 3×10\hat{ }6 CFU/ml for C. lusitaniae [Guinea J. et al., Rapid antifungal susceptibility determination for yeast isolates by use of Etest performed directly on blood samples from patients with fungemia, J Clin Microbiol. 2010 June, 48(6): 2205-12]. Then, the sensitivity test vs the two oils has been performed. The disk diffusion method was carried out as follows: for each tested strain, 4 plates of Potato Dextrose Agar (PDA) were seeded in 3 directions to produce a confluent growth, by sterile swab soaked in the cell suspension. Sorbent paper disks (Oxoid Thermofisher SpA, Milan, Italy) 6.0 mm diameter were placed on the agar surface and added with 10 μl of each essential oil. Disks soaked with 10 μg amphotericin B were used as positive control. Also disks of chlortrimazole (from 10 μg to 40 μg) were used, but the inhibition rings could not be clearly read due to the formation of a re-growth ring. Disks soaked with 10 μl pure dimethylsulphoxide (DMSO, Sigma-Aldrich, St. Louis, MO, USA) were used as negative control. The plates were incubated at 35° C. for 24 hours. All the experiments were carried out in duplicate. The inhibiting effect of the oils were considered to be effective when the inhibition ring was equal or greater than that caused by amphotericin B. For amphotericin B, an inhibition zone (IZ)≥15 mm was considered as “sensitivity”, from 10 to 14 mm as “intermediate sensitivity” and <10 mm as “resistance” [Kauser et al., Agreement of Direct Antifungal Susceptibility Testing from Positive Blood Culture Bottles with Conventional Method for Candida Species, J Clin Microbiol. 2016 February, 54(2):343-34].

The reading scheme of disk diffusion is shown in FIG. 1.

### Antifungal Activity: Method in Microdilution

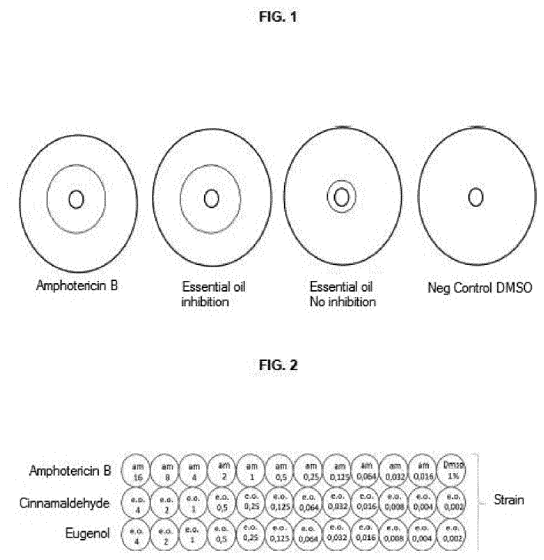

The antifungal activity of amphotericin B and of cinnamaldheyde and eugenol were tested with the microdilution method as reported by Bona et al. [Bona et al., Sensitivity of Candida albicans to essential oils: are they an alternative to antifungal agents? J Appl Microbiol. 2016 December, 121(6): 1530-1545] with small changes. All the 18 Candida spp strains were tested. For each isolated, the colonies deriving from cultures (24 hours) in PDA were diluted in saline up to a concentration of 2×10\hat{ }3 CFU/ml (so obtaining a final concentration of 10 3CGU/ml). Cinnamaldehyde and eugenol were dissolved in DMSO (80% oil, 20% DMSO), then diluted in Sabouraud broth up to a concentration of 8% v/v (so obtaining a final concentration of 4% v/v). Then, in a 96-well microtiter plate ½ serial dilution of amphotericin B (range 32-0.032 μg/ml, for a final range of 16-0.016 μg/ml) and of the essential oils (range 8-0.004% v/v, for a final range of 4-0.002% v/v) were carried out. Each well was then injected with 100 μl of cell suspension. The negative control contained DMSO diluted in Sabouraud broth (at 2% v/v, for a final concentration of 1% v/v). The plates were incubated at 35° C. for 48 hours. Each experiment was carried out in duplicate. The Minimum Inhibitory Concentration (MIC) was determined as the concentration of the oils in the first well which is not cloudy. The Minimum Fungicidal Concentration (MFC) was determined as the lowest concentration of the oils, with the agar culture showing the complete absence of fungal growth. The determination of the amphotericin B MIC was carried out also according to the EUCAST guidelines (cutoff 1 μg/ml) [https://eucast.org/astoffungi/clinicalbreakpointsforantifungals], all the results being consistent. The reading scheme of the microdilution method is reported in FIG. 2.

Antifungal Activity: Time-Kill Curve

A C. albicans strain (C.a. 15) was incubated with cinnamaldheyde and eugenol at their MFC. The C. albicans colonies were suspended in Sabouraud broth at a density corresponding to #1.4 McFarland (4×10\hat{ }6 CFU/ml), then diluted 1/10 (4×10\hat{ }5 CFU/ml, for a final concentration of 2×10\hat{ }5 CFU/ml). The suspension was divided into aliquots, two aliquots were incubated with the oils (½ v/v) prepared at a concentration double of their MFC (for a final concentration equal to MFC); the third aliquot was diluted ½ with Sabouraud broth and was used as negative control. The mixtures were incubated at 35° C. on swinging plate. At T0, T1 h, T2 h, T4 h, T6 h, T8 h, T12 h and T24 h, 1/10 serial dilutions were carried out, to determine the total vital count (TVC). From each dilution, 100 μl were seeded in PDA and incubated at 35° C. for 24 hours, then the colonies were counted, their number expressed as CFU/ml. The time-kill curve method was carried out according to the scheme reported in FIG. 3. The test was carried out in duplicate.

### Synergic Effect: Checkerboard

To evaluate the eventual synergic effect of the oils in combination the checkerboard method on 96-well microtiter plate was used, as depicted in the following Table 1:

TABLE 1
Checkerboard

| | 1 | 2 | 3 | 4 | 5 | 6 | 7 | 8 | 9 | 10 | 11 | 12 |
|---|---|---|---|---|---|---|---|---|---|---|---|---|
| a | CNTR | 0.008A | 0.016A | 0.031A | 0.0625A | 0.125A | 0.25A | 0.5A | A | 2A | 4A | 8A |
| b | 0.0625B | 0.008A + | 0.016A + | 0.031A + | 0.0625A + | 0.125A + | 0.25A + | 0.5A + | A + | 2A + | 4A + | 8A + |
| | | 0.0625B | 0.0625B | 0.0625B | 0.0625B | 0.0625B | 0.0625B | 0.0625B | 0.0625B | 0.0625B | 0.0625B | 0.0625B |
| c | 0.125B | 0.008A + | 0.016A + | 0.031A + | 0.0625A + | 0.125A + | 0.25A + | 0.5A + | A + | 2A + | 4A + | 8A + |
| | | 0.125B | 0.125B | 0.125B | 0.125B | 0.125B | 0.125B | 0.125B | 0.125B | 0.125B | 0.125B | 0.125B |
| d | 0.25B | 0.008A + | 0.016A + | 0.031A + | 0.0625A + | 0.125A+ | 0.25A + | 0.5A + | A + | 2A + | 4A + | 8A + |
| | | 0.25B | 0.25B | 0.25B | 0.25B | 0.25B | 0.25B | 0.25B | 0.25B | 0.25B | 0.25B | 0.25B |
| e | 0.5B | 0.008A + | 0.016A + | 0.031A + | 0.0625A + | 0.125A + | 0.25A + | 0.5A + | A + | 2A + | 4A + | 8A + |
| | | 0.5B | 0.5B | 0.5B | 0.5B | 0.5B | 0.5B | 0.5B | 0.5B | 0.5B | 0.5B | 0.5B |
| f | B | 0.008A + | 0.016A + | 0.031A + | 0.0625A + | 0.125A + | 0.25A + | 0.5A + | A + | 2A + | 4A + | 8A + |
| | | B | B | B | B | B | B | B | B | B | B | B |
| g | 2B | 0.008A + | 0.016A + | 0.031A + | 0.0625A + | 0.125A + | 0.25A + | 0.5A + | A + | 2A + | 4A + | 8A + |
| | | 2B | 2B | 2B | 2B | 2B | 2B | 2B | 2B | 2B | 2B | 2B |
| h | 4B | 0.008A + | 0.016A + | 0.031A + | 0.0625A + | 0.125A + | 0.25A + | 0.5A + | A + | 2A + | 4A + | 8A + |
| | | 4B | 4B | 4B | 48 | 4B | 4B | 4B | 4B | 4B | 4B | 4B |

A: cinnamaldheyde-B: eugenol

100 μl Sabouraud broth were put into all wells except H12. 200 μl cinnamaldheyde were added to well H12 and 100 μl of the same oil (at a concentration corresponding to 32×MIC, for a final concentration corresponding to 8×MIC) were added to wells from A12 to G12; a ½ dilution in horizontal direction was carried out, up to column 2. 100 μl eugenol (at a concentration corresponding to 16×MIC, for a final concentration corresponding to 4×MIC) were added to wells from H1 to H12, then the second dilution, in vertical direction, was carried out, up to row B. Colonies di C. albicans (C.a. 15 strain) derived from a 24-hour culture in PDA were suspended in Sabouraud broth at the concentration of 2×10\hat{ }3 CFU/ml (for a final concentration of 10\hat{ }3 CFU/ml). 100 μl of the suspension were put into all wells. The plates were incubated at 35° C. for 48 hours. MICs were determined as for the microdilution method. Well A1 was used as negative control. The test was carried out in duplicate.

FICI (Fractional Inhibitory Concentration Index) was determined as follows:

FICI=MIC (cinnamaldheyde in combination)/MIC (cinnamaldheyde)+MIC (eugenol in combination)/MIC (eugenol)

A synergic effect is found when the FICI value is ≤0.5; an additive effect when 0.5<FICI≤1; an indifferent effect when 1<FICI≤4 and an antagonistic effect when FICI>4 [Vuuren et al., Antimicrobial activity of limonene enantiomers and 1,8-cineole alone and in combination, Flavour and Fragrance Journal, 2007, 22(6):540-544; Stanojevic et al., In vitro synergistic antibacterial activity of Salvia officinalis L. and some preservatives, Archives of Biological Sciences Belgrade, 2010, 62(1): 175-83].

### Effects on Lactobacillus Acidophilus

The antibacterial activity of cinnamaldheyde and eugenol vs L. acidophilus was studied by using the disk diffusion method (reading scheme as reported in FIG. 1) and the microdilution method (reading scheme as reported in FIG. 2). A L. acidophilus strain named La-14 (Danisco SpA, Milan, Italy) was tested.

### Disk Diffusion

Colonies deriving from a 24-hour culture in DeManRogosaSharpe agar (MRS, OXOID SpA, Milan, Italy) were suspended in saline up to a concentration corresponding to #0.5 McFarland. A total of 3 plates of MRS agar were seeded in 3 directions to produce a confluent growth, by sterile swab soaked in the bacterial suspension. Sorbent paper disks 6.0 mm diameter were placed on each plate and soaked with 10 μl of each oil. Disks soaked with 10 μg pure DMSO were used as negative control. The plates were incubated at 37° C. for 24 hours in anaerobiosis (anaerogen gaspack, OXOID SpA, Milan, Italy). The experiment was carried out in duplicate.

### Microdilution

The essential oils (80% oil, 20% DMSO) were dissolved in MRS broth up to a concentration of 8% v/v (so obtaining a final concentration of 4% v/v). In a 96-well microtiter plate, ½ serial dilutions of the oils were carried out (volume: 100 μl. Range 8-0.004% v/v, for a final range 4-0.002% v/v). Each well was then injected with 100 μl of the cell suspension. The microtiter plates were incubated at 37° C. for 48 hours in anaerobiosis. MIC was determined as the concentration of the oils in the first well which is not cloudy. The experiment was carried out in duplicate.

### Results

### Disk Diffusion

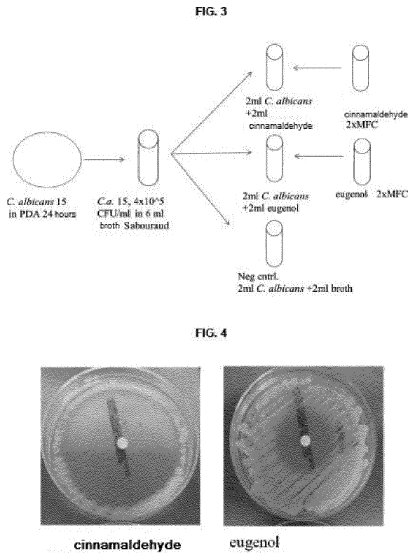

Eighteen strains belonging to Candida spp (15 C. albicans, 2 C. glabrata and 1 C. lusitaniae) were tested with the disk diffusion method. The inhibiting effect of the oils was considered to be effective when the inhibition ring resulted to be equal or greater than that caused by amphotericin B. Cinnamaldheyde and eugenol inhibited all tested strains. The results are summarized in the following Tables 2 and 3 and in FIG. 4.

TABLE 2
Distribution of the inhibition rings (including 6.0 mm of the disk)

| | diameter (mm) | % inhibited strains |
|---|---|---|
| Amphotericin B | | |
| average | 15.4 | |
| range | 10-20.5 | |
| standard deviation | 2.7 | |
| Cinnamaldheyde | | 100% (18/18) |
| average | 69 | |
| range | 55-75 | |
| standard deviation | 6 | |
| Eugenol | | 100% (18/18) |
| average | 35.2 | |
| range | 30.5-41 | |
| standard deviation | 3.5 | |
| Neg control (DMSO) | // | |

TABLE 3
Diameters (mm) of the inhibition rings
(including 6.0 mm of the disk)

| | Amphotericin B | Cinnamaldheyde | Eugenol |
|---|---|---|---|
| C. albicans 1 | 13.0 | 65.0 | 35.0 |
| C. albicans 2 | 15.5 | 70.0 | 35.0 |
| C. albicans 3 | 16.0 | 67.0 | 33.0 |
| C. albicans 4 | 19.5 | 66.0 | 35.0 |
| C. albicans 5 | 16.0 | 71.0 | 35.0 |
| C. albicans 6 | 16.0 | 67.0 | 36.0 |
| C. albicans 7 | 16.0 | 75.0 | 44.0 |
| C. albicans 8 | 20.5 | 67.0 | 41.0 |
| C. albicans 9 | 12.0 | 70.5 | 34.0 |
| C. albicans 10 | 15.0 | 72.5 | 34.5 |
| C. albicans 11 | 14.5 | 75.0 | 34.0 |
| C. albicans 12 | 16.5 | 75.0 | 34.0 |
| C. albicans 13 | 16.0 | 71.0 | 35.0 |
| C. albicans 14 | 19.0 | 75.0 | 34.0 |
| C. albicans 15 | 15.5 | 70.0 | 34.0 |
| C. glabrata 1 | 15.0 | 55.0 | 35.0 |
| C. glabrata 2 | 11.0 | 55.0 | 30.5 |
| C. lusitaniae 1 | 10.0 | 75.0 | 43.0 |

### Microdilution

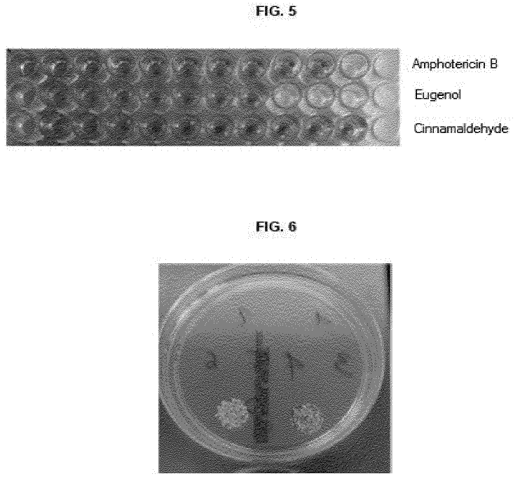

The antifungal activity of cinnamaldheyde and eugenol was further analyzed by using the microdilution method. All the inhibition rings of amphotericin B detected by the disk diffusion method fall within the “sensitivity” or “intermediate sensitivity” category, also the microdilution confirmed these results. 1% DMSO (negative control) did not inhibit the cell growth. Cinnamaldheyde showed lower MICs and MFCs, even so eugenol MICs and MFCs are very low. The results of the test in microdilution are reported in Tables 4 and 5. In FIGS. 5 and 6, examples of the MIC and MFC readings are reported.

TABLE 4
distribution of MICs and MFCs

| | MIC | MIC | | MFC | MFC | |
|---|---|---|---|---|---|---|
| | aver- | st. | MIC | aver- | St. | MFC |
| | age | dev. | range | age | dev. | range |
| Ampho- | 0.052 | 0.04 | ≤0.016- | 0.11 | 0.08 | ≤0.016- |
| tericin B | | | 0.125 | | | 0.25 |
| (μg/ml) | | | | | | |
| Cinnam- | 0.0048 | 0.0042 | ≤0.002- | 0.01 | 0.015 | ≤0.002- |
| aldheyde | | | 0.064 | | | 0.64 |
| (% v/v) | | | | | | |
| Eugenol | 0.043 | 0.033 | 0.008- | 0.067 | 0.035 | 0.016- |
| (% v/v) | | | 0.125 | | | 0.125 |

TABLE 5
reading of MICs and MFCs in microdilution

| | Amphotericin B | | Cinnamaldheyde | | Eugenol | |
|---|---|---|---|---|---|---|
| | MIC | MFC | MIC | MFC | MIC | MFC |
| | (μg/ml) | (μg/ml) | (% v/v) | (% v/v) | (% v/v) | (% v/v) |
| C. albicans 1 | 0.032 | 0.064 | 0.004 | 0.008 | 0.032 | 0.032 |
| C. albicans 2 | 0.032 | 0.064 | 0.004 | 0.008 | 0.032 | 0.032 |
| C. albicans 3 | 0.032 | 0.064 | 0.004 | 0.004 | 0.016 | 0.032 |
| C. albicans 4 | 0.032 | 0.125 | ≤0.002 | 0.008 | 0.064 | 0.064 |
| C. albicans 5 | 0.016 | 0.250 | 0.004 | 0.004 | 0.008 | 0.032 |
| C. albicans 6 | 0.032 | 0.064 | 0.004 | 0.008 | 0.032 | 0.032 |
| C. albicans 7 | 0.032 | 0.064 | ≤0.002 | 0.008 | 0.032 | 0.064 |
| C. albicans 8 | 0.016 | 0.032 | 0.004 | 0.008 | 0.032 | 0.032 |
| C. albicans 9 | 0.125 | 0.250 | 0.016 | 0.064 | 0.125 | 0.125 |
| C. albicans 10 | 0.032 | 0.032 | ≤0.002 | ≤0.002 | 0.016 | 0.016 |
| C. albicans 11 | 0.125 | 0.250 | ≤0.002 | ≤0.002 | 0.064 | 0.125 |
| C. albicans 12 | ≤0.016 | 0.016 | ≤0.002 | ≤0.002 | 0.016 | 0.064 |
| C. albicans 13 | 0.064 | 0.125 | 0.008 | 0.016 | 0.064 | 0.125 |
| C. albicans 14 | 0.032 | 0.125 | 0.004 | 0.016 | 0.032 | 0.125 |
| C. albicans 15 | 0.032 | 0.064 | 0.004 | 0.008 | 0.032 | 0.064 |
| C. glabrata 1 | 0.032 | 0.064 | 0.004 | 0.008 | 0.032 | 0.032 |
| C. glabrata 2 | 0.125 | 0.125 | 0.016 | 0.016 | 0.125 | 0.125 |
| C. lusitaniae 1 | 0.125 | 0.250 | ≤0.002 | ≤0.002 | 0.032 | 0.064 |

### Time-Kill Curve

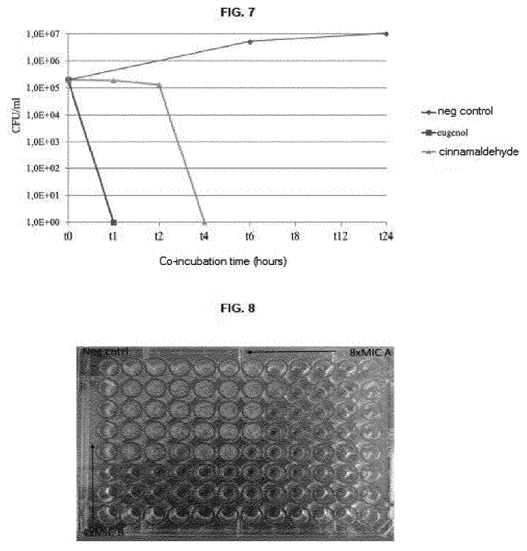

2×10\hat{ }5 CFU/ml of C.a. 15 strain were co-incubated with cinnamaldheyde and eugenol. At T0, T1 h, T2 h, T4 h, T6 h, T8 h, T12 h and T24 h, the total vital count was determined. Eugenol killed all the fungal cells within the first hour of co-incubation. When the C. albicans cells were incubated with cinnamaldheyde, the vital count remained stable for 2 hours, at 4 hours there were no vital cells. The results are reported in FIG. 7.

### Synergic Effect

By testing cinnamaldheyde in combination with eugenol, the first non-cloudy well resulted to be C8, wherein the cinnamaldheyde MIC is ½ of the starting MIC and the eugenol MIC is ⅛ of the starting MIC. The FICI value results to be 0.625, corresponding to an addictive effect, very close to a synergic effect (FIG. 8).

### Effects on Lactobacillus Acidophilus

Lactobacillus acidophilus is the main component (90%) of the Doderlein vaginal complex, forming a protective biofilm on the mucosa and inhibiting the overgrowth of other noxious microorganisms [J. P. Lepargneur, V. Rousseau, Protective role of Doderlein flora, Journal de Gynécologie Obstétrique et Biologie de la Reproduction 31(5):485-94]. It is extremely important that the antimicrobial substances used at vaginal level do not affect the growth of the normal vaginal microflora.

By the disk diffusion method, eugenol did not produced any inhibition ring, cinnamaldehyde produced a small ring 10 mm diameter. By evaluating the activity of cinnamaldehyde in broth, the MIC vs L. acidophilus resulted to be 0.032% v/v, this MIC being the double of the observed maximum MIC (in 2 strains out of 18 only) vs Candida spp and about 6.7 fold higher than the average MIC. By evaluating the activity of eugenol in broth, the MIC vs L. acidophilus resulted to be 0.25% v/v, this MIC being the double of the observed maximum MIC (in 2 strains out of 18 only) vs Candida spp and about 6 fold higher than the average MIC. From these observations it is clear how the use of these oils at the concentrations needed to treat vaginal candidosis allows to maintain unaltered the normal bacterial flora.

1: A combination of cinnamaldehyde and eugenol for use as antimycotic in humans and animals for the treatment of fungal infections caused by Candida, without affecting the growth of Lactobacillus acidophilus, wherein cinnamaldehyde and eugenol are in a ratio ranging from 1:1 to 1:10 w/w.

2: A composition containing the combination according to claim 1 in admixture with a suitable carrier for topical or systemic administration.

3: A combination according to claim 1 wherein the daily dose of cinnamaldehyde ranges from 0.03 mg/kg to 300 mg/kg and the daily dose of eugenol ranges from 0.03 mg/kg to 300 mg/kg.

4: A combination according to claim 1 wherein the cinnamaldehyde:eugenol ratio is 1:2 w/w.

5: A combination according to claim 1 wherein cinnamaldehyde and eugenol are used in the form of essential oils.

6: A combination according to claim 5 wherein the essential oils are selected from cinnamon essential oil and clove essential oil.

7: A combination according to claim 1 wherein cinnamaldehyde and eugenol are used in purified form.

8: A combination according to claim 1 for use against fungal infections caused by Candida, wherein the fungal infections are candidosis or thrush.

9: A method of treating fungal infections caused by Candida, comprising the step of administering a combination of cinnamaldehyde and eugenol to treat a fungal infection of a human or animal, wherein the cinnamaldehyde and eugenol are in a ratio ranging from 1:1 to 1:10 w/w.

10: The method according to claim 9, wherein the combination is contained within in a composition of a mixture of the combination with a carrier and said administering step is provided by topical or systemic administration.

11: The method according to claim 9, wherein said administering step includes a daily dose of cinnamaldehyde ranging from 0.03 mg/kg to 300 mg/kg and a daily dose of eugenol from 0.03 mg/kg to 300 mg/kg.

12: The method according to claim 9, wherein the cinnamaldehyde:eugenol ratio is 1:2 w/w.

13: The method according to claim 9, wherein the cinnamaldehyde and eugenol are in the form of essential oils.

14: The method according to claim 13, wherein the essential oils are selected from cinnamon essential oil and clove essential oil.

15: The method according to claim 9, wherein the cinnamaldehyde and eugenol are in purified form.

16: The method according to claim 9, wherein the fungal infections are candidosis or thrush.
